(19) United States
(12) Patent Application Publication
(10) Pub. No.: US 2026/0065430 A1
(43) Pub. Date: Mar. 5, 2026

(54) DENOISING DIFFUSION MODELS FOR PLUG-AND-PLAY MR IMAGE RESTORATION/RECONSTRUCTION

(71) Applicant: Siemens Healthineers AG, Forchheim (DE)

(72) Inventors: Mahmoud Mostapha, Princeton, NJ (US); Radu Miron, Iasi (RO); Mariappan S. Nadar, Plainsboro, NJ (US); Boris Mailhe, Plainsboro, NJ (US); Nirmal Janardhanan, Monmouth Junction, NJ (US); Rainer Schneider, Höchstadt (DE); David Grodzki, Erlangen (DE); Till Hülnhagen, Erlangen (DE); Tobias Würfl, Erlangen (DE); Omar Darwish, London (GB)

(21) Appl. No.: 19/057,174

(22) Filed: Feb. 19, 2025

Related U.S. Application Data
(60) Provisional application No. 63/687,799, filed on Aug. 28, 2024.

(30) Foreign Application Priority Data
Aug. 28, 2024 (EP) 24465563.5

(51) Int. Cl. G06T 5/60; G06T 5/70

(52) U.S. Cl. CPC G06T 5/60; G06T 5/70; G06T 2207/10088; G06T 2207/20081; G06T 2207/20084; G06T 2207/30004

(57) ABSTRACT

Systems and methods for image restoration and reconstruction using diffusion models. A diffusion plug and play model includes measurement during reverse diffusion steps, which is based on DDIM and supports fast sampling. This measurement is carried out after a correction step that accounts for the inaccurate estimation resulting from computing the proximal solution.

### CROSS REFERENCE TO RELATED APPLICATIONS

This application claims priority to U.S. provisional application Ser. No. 63/687,799, filed Aug. 28, 2024, and European Patent Application EP24465563.5, filed Aug. 28, 2024, both of which are entirely incorporated by reference.

### FIELD

This disclosure relates to medical imaging.

### BACKGROUND

Magnetic resonance imaging, or MRI, is a noninvasive medical imaging test that can generate detailed images of almost every internal structure in the human body, including, for example organs, bones, muscles, and blood vessels. Traditional MRI is slow due to the need for sequential data acquisition, often resulting in long scan times that can cause patient discomfort and motion artifacts. Acceleration techniques aim to overcome these limitations using methods like parallel imaging, compressed sensing, and deep learning-based reconstructions. Parallel imaging, such as SENSE and GRAPPA, utilizes multiple receiver coils to acquire data simultaneously, reducing scan time. Compressed sensing leverages sparsity in MRI images to reconstruct high-quality images from undersampled data, significantly shortening acquisition time. Deep learning-based approaches have been proposed that use neural networks to enhance image reconstruction, improving speed and accuracy.

In particular, deep learning approaches referred to as Plug-and-play (PnP) have been recently used that rely on pre-trained denoisers to address various imaging tasks without the need to train specific models for each task. However, current PnP image restoration and reconstruction methods heavily depend on discriminative Gaussian denoisers, that can exhibit unstable behaviors, impacting their versatility and resulting in a suboptimal quality of reconstructed images. Despite the exceptional performance of diffusion models in high-quality image synthesis, their potential as a generative denoiser before plug-and-play image restoration methods still needs to be improved particularly for complex MRI data. Current attempts to apply diffusion models for image restoration either fail to achieve satisfactory results or typically require an unacceptable number of Neural Function Evaluations (NFEs) during inference.

### SUMMARY

By way of introduction, the preferred embodiments described below include methods, systems, instructions, and/or computer readable media for magnetic resonance imaging and image reconstruction using denoising diffusion models for plug-and-play MR image restoration/reconstruction.

In a first aspect, a method for diffusion plug and play (PnP) image reconstruction of medical imaging data, the method comprising: acquiring medical imaging data of a portion of a patient; iteratively refining the medical imaging data using diffusion PnP, wherein for each step of an iterative process, a pretrained diffusion model is used to remove noise to predict a next state of the iterative process and measurement data is incorporated by solving a data proximal subproblem, the measurement data applied to the next state to ensure consistency; and outputting a reconstructed medical image of the portion of the patient.

In a second aspect, a system for diffusion plug and play (PnP) image reconstruction of medical imaging data, the system comprising: a medical imaging device configured to acquire MR data; a memory configured to store a model configured to reconstruct an MR image from the MR data, wherein the model is configured to iteratively refine the MR data using diffusion PnP, wherein for each step of the iterative process, a pretrained diffusion model is used to remove noise to predict a next state of the iterative process and measurement data is incorporated by solving a data proximal subproblem, the measurement data applied to the next state to ensure consistency; and a processor configured to reconstruct and/or restore an MR image from the MR data using the model.

In a third aspect, a method for diffusion plug and play (PnP) image restoration of medical imaging data, comprising: acquiring medical imaging data; iteratively restoring the medical imaging data using a diffusion PnP model that includes measurement during reverse diffusion steps, wherein the measurement is carried out after a correction step that accounts for the inaccurate estimation resulting from computing a proximal solution; and outputting a restored image.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 depicts an example system for magnetic resonance imaging and image reconstruction using denoising diffusion models for plug-and-play MR image restoration/reconstruction according to an embodiment.

FIG. 2 depicts an example of a diffusion process.

FIG. 3 depicts hallucinations created by generative processes.

FIG. 4 depicts an example workflow for magnetic resonance imaging and image reconstruction using denoising diffusion models for plug-and-play MR image restoration/reconstruction according to an embodiment.

FIG. 5 depicts an example of one step for image reconstruction using denoising diffusion models for plug-and-play MR image restoration/reconstruction according to an embodiment.

FIG. 6 depicts an example algorithm for image reconstruction using denoising diffusion models for plug-and-play MR image restoration/reconstruction according to an embodiment.

FIG. 7 depicts an example workflow for training a diffusion model according to an embodiment.

FIG. 8 depicts an example U-net architecture.

FIG. 9 depicts example outputs of a diffusion model.

FIG. 10 depicts an example artificial neural network.

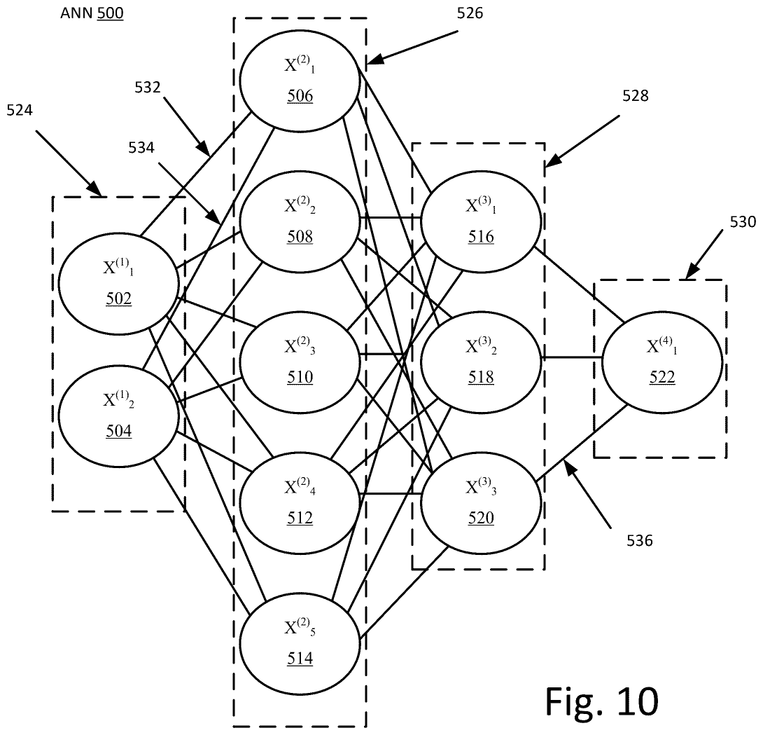

FIG. 11 depicts an example convolutional neural network.

### DETAILED DESCRIPTION

Embodiments described herein provide systems and methods that combines a plug-and-play method with a diffusion sampling framework to accurately restore complex MRI data regarding reconstruction faithfulness and perceptual quality.

MRI is a widely used medical imaging technique that provides high-resolution anatomical and functional imaging without ionizing radiation. However, MRI acquisitions are often slow, leading to long scan times and patient discomfort. To address this issue, researchers have explored deep learning-based methods for accelerating MRI reconstruction from undersampled data which may be acquired in shorter imaging sessions. One approach is the use of Denoising Diffusion Probabilistic Models (DDPMs), a class of generative models that have demonstrated state-of-the-art performance in image synthesis and restoration tasks. While diffusion models have shown better generative ability than GAN and VAE, their slow inference speed limits their use in clinical applications.

Embodiments described herein provide a plug-and-play (PnP) method with a diffusion sampling framework to reconstruct/restore medical images. Embodiments adapt the quadratic sequence from Denoising Diffusion Implicit Models (DDIM) for sampling, which has more sampling steps at low-noise regions. This allows for the sampling sequence (length T) to be a subset of N used in training the diffusion prior. As a result, embodiments using the described diffusion PnP method may produce detailed images with less than 100 NFEs allowing for increased speed while maintaining accuracy. Embodiments further provide for a wide range of IR tasks of complex-valued MRI images as well as accommodating to different user preferences for the reconstructed image impression through tuning Diffusion PnP hyperparameters, for example X and (that control the strength of the condition guidance and the level of noise injected at each timestep.

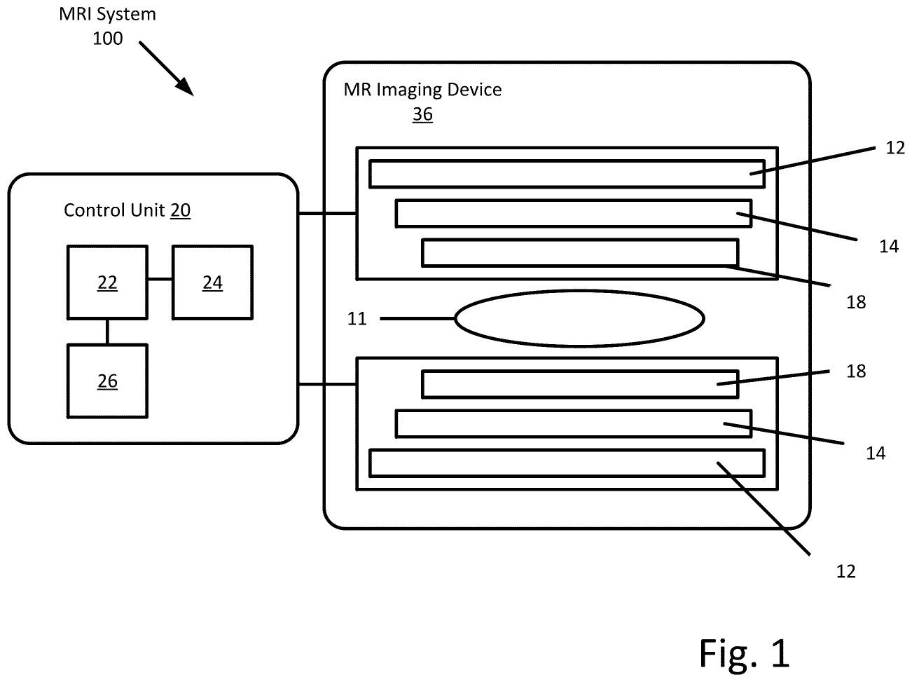

FIG. 1 depicts an example MR system 100 for magnetic resonance imaging and image reconstruction using denoising diffusion models for plug-and-play MR image restoration/reconstruction. MRI is a noninvasive medical imaging procedure for imaging internal structures in the human body, for example, organs, bones, muscles, and blood vessels. The examples described herein use a magnetic resonance (MR) context (i.e., a magnetic resonance scanner), but the reconstruction techniques and models may be used for other medical imaging procedures such as computed tomography (CT) or positron emission tomography (PET) where applicable. The examples may further use knee and brain MRI procedures as examples, but any organ or region may be imaged by the system 100. The system 100 use a generative diffusion model(s) to reconstruct/restore (denoise, upscale, refine etc.) an image from medical imaging data acquired by an MR scanner 36 of a patient 11. The MR system 100 includes an MR imaging device 36, control unit 20, and/or a server. The MR imaging device 36 is only exemplary, and a variety of MR scanning systems may be used to collect the MR data. The MR imaging device 36 (also referred to as a MR scanner or image scanner) is configured to scan the patient 11 to provide k-space measurements (measurements in the frequency domain). The MR system 100 further includes a control unit 20 that is configured to process the MR signals and generate (reconstruct, restore) images of the object or patient 11 for display to an operator or further analysis. The control unit 20 includes a processor 22 that is configured to execute instructions, or the method described herein. The control unit 20 may store the MR signals and images in a memory 24 for later processing or viewing. The control unit 20 may include a display 26 for presentation of images to an operator.

In the MR system 100, magnetic coils 12 create a static base or main magnetic field B0 in the body of patient 11 or an object positioned on a table and imaged. Within the magnet system are gradient coils 14 for producing position dependent magnetic field gradients superimposed on the static magnetic field. Gradient coils 14, in response to gradient signals supplied thereto by a gradient and control unit 20, produce position dependent and shimmed magnetic field gradients in three orthogonal directions and generate magnetic field pulse sequences. The shimmed gradients compensate for inhomogeneity and variability in an MR imaging device magnetic field resulting from patient anatomical variation and other sources. The control unit 20 may include a RF (radio frequency) module that provides RF pulse signals to RF coil 18. The RF coil 18 produces magnetic field pulses that rotate the spins of the protons in the imaged body of the patient 11 by ninety degrees or by one hundred and eighty degrees for so-called “spin echo” imaging, or by angles less than or equal to 90 degrees for “gradient echo” imaging. Gradient and shim coil control modules in conjunction with RF module, as directed by control unit 20, control slice-selection, phase-encoding, readout gradient magnetic fields, radio frequency transmission, and magnetic resonance signal detection, to acquire magnetic resonance signals representing planar slices of the patient 11. In response to applied RF pulse signals, the RF coil 18 receives MR signals, e.g., signals from the excited protons within the body as the protons return to an equilibrium position established by the static and gradient magnetic fields. The MR signals are detected and processed by a detector within RF module and the control unit 20 to provide an MR dataset to a processor 22 for processing into an image.

In some embodiments, the processor 22 is located in the control unit 20, in other embodiments, the processor 22 is located remotely. A two or three-dimensional k-space storage array of individual data elements in a memory 24 of the control unit 20 stores corresponding individual frequency components including an MR dataset. The k-space array of individual data elements includes a designated center, and individual data elements individually include a radius to the designated center. The magnetic field generator (including coils 12, 14 and 18) generates a magnetic field for use in acquiring multiple individual frequency components corresponding to individual data elements in the storage array. A storage processor in the control unit 20 stores individual frequency components acquired using the magnetic field in corresponding individual data elements in the array. The row and/or column of corresponding individual data elements alternately increases and decreases as multiple sequential individual frequency components are acquired. The magnetic field generator acquires individual frequency components in an order corresponding to a sequence of substantially adjacent individual data elements in the array, and magnetic field gradient change between successively acquired frequency components is substantially minimized.

During an imaging procedure, the MR imaging device 36 is configured by the imaging protocol to scan a region of a patient 11. For example, in MR, such protocols for scanning a patient 11 for a given examination or appointment include diffusion-weighted imaging (acquisition of multiple b-values, averages, and/or diffusion directions), turbo-spin-echo imaging (acquisition of multiple averages), and/or contrast. In one embodiment, the protocol is for compressed sensing. The control unit 20 is configured to reconstruct an image using the acquired MRI data from an imaging procedure. Image reconstruction may be performed by the system 100 or other computing devices. MRI image reconstruction is the process of converting raw data from an MRI scan into a clinical image. It is a critical step in the MRI process, as the quality of the reconstructed image can affect the accuracy of the diagnosis. In particular, when using accelerated MRI, the reconstruction/restoration of the MRI images is important as the acquired imaging data may be sparse and thus provide an initial image/data that is, for example, noisy. Noisy images may be difficult to interpret and may result in inaccurate diagnoses. The control unit 20 may be configured to restore a reconstructed image.

In embodiments described herein, image reconstruction/restoration uses a generative deep learning framework, for example diffusion models, for reconstructing/restoring images from acquired MRI data. The generative deep learning model utilizes prior knowledge either with (supervised) or without (unsupervised) knowledge of a specific reconstruction task. By decoupling learning of the prior knowledge from the reconstruction task, the diffusion models may overcome existing issues of costly training and poor robustness to varied scan parameters.

MRI acquisitions may be slow, for example requiring long scan times and patient discomfort. To address this issue, accelerated scans have been used that take less time by acquiring less data. The resulting sparse data may be reconstructed/restored using deep learning generative methods. Diffusion models (DMs) are a class of deep generative neural networks configured to sample from an unknown data distribution. One known diffusion model implementation is referred to as a Denoising Diffusion Probabilistic Model (DDPM). DDPMs are a type of generative model that learn to generate complex data distributions by iteratively refining noisy samples. Diffusion models include a forward diffusion process and a reverse diffusion process. In the forward diffusion process noise is added to an image over multiple time steps, effectively transforming it into a random noise distribution. In the reverse diffusion process, the model denoises the noisy image step by step, effectively recovering the original data distribution. In MRI reconstruction, DDPMs leverage these processes to generate high-fidelity images from undersampled k-space data and/or restore noisy or otherwise deficient images.

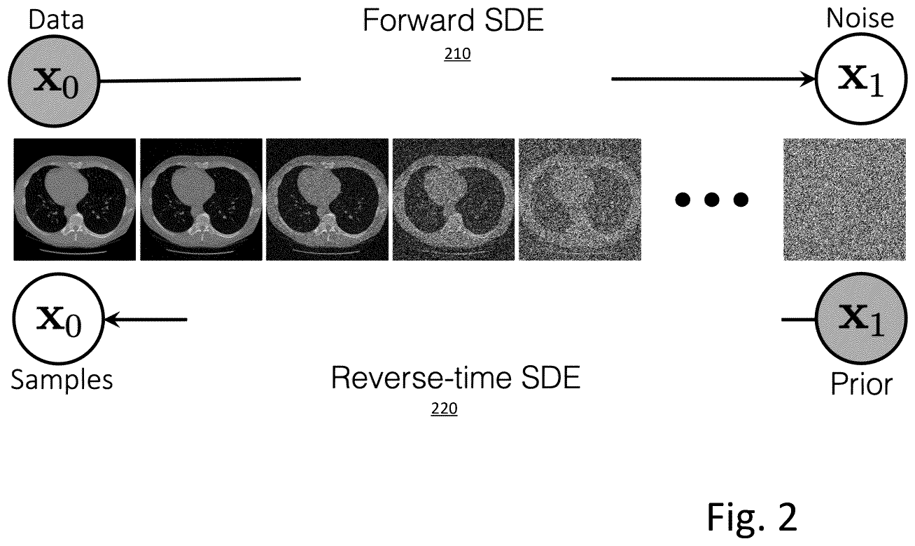

FIG. 2 depicts an example of a generative diffusion process for image processing including the forward process 210 and the reverse process 220 (also referred to as the inference stage). The goal of the diffusion model is to learn the diffusion process for a given dataset, such that the process can generate new elements that are distributed similarly as the original dataset. In the forward stochastic differential equation (SDE) noise is added to the input image over and over again until the image is practically all noise. At each step, the diffusion model learns how to map images to their corresponding noise-free measurements. In the reverse step, the learned diffusion model is used to recover the data by reversing this noising process. Image reconstruction in MRI is a similar inverse problem that attempts to find an image from noisy scan measurements. To solve the inverse problem a forward model is defined that maps noisy MR images to their corresponding noise-free measurements. As measurements become noisier (for example as scan time is reduced) or less complete (for example when using increased acceleration), the resulting image reconstruction problem becomes highly ill-posed, meaning it has no stable, unique solution. In such situations the acquired measurements are said to be sparse, i.e., they are generally insufficient to uniquely specify a finite-dimensional approximation of the sought-after object, even in the absence of measurement noise or errors related to modeling the imaging system. False structures may arise due to the reconstruction method incorrectly estimating parts of the object that either did not contribute to the observed measurement data or cannot be recovered in a stable manner, a phenomenon that is referred to as hallucinations.

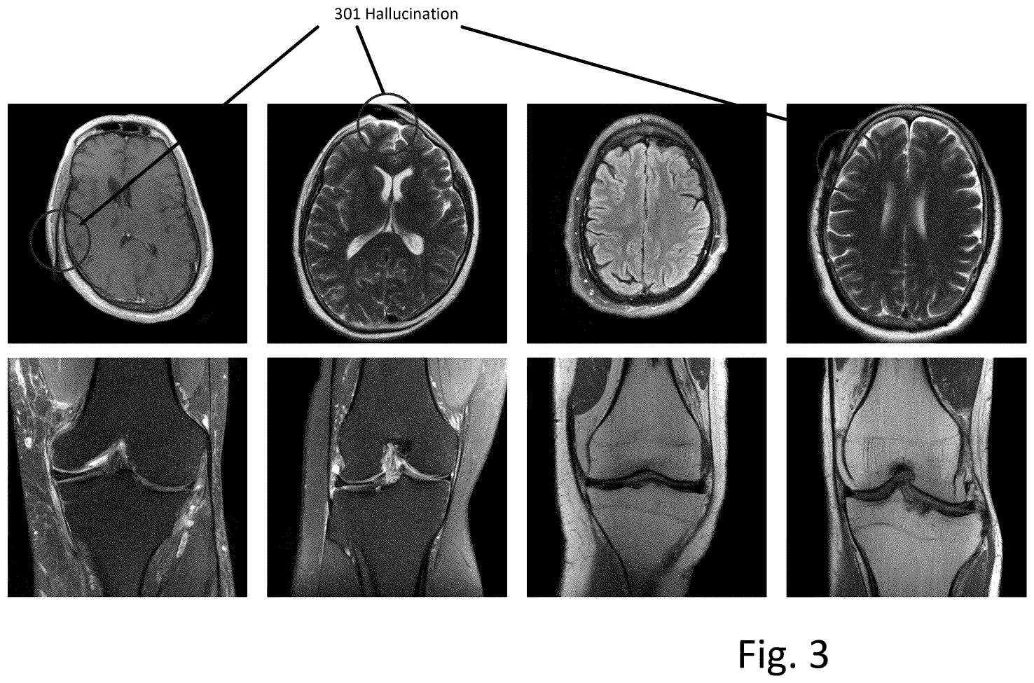

FIG. 3 depicts various hallucinations 301 in MRI images. For example, in the brain MRI images, the bone structure is poorly generating leading to gaps in the structure. While these errors are obvious, less pronounced hallucinations may lead to poor diagnostics or analysis where it may be difficult to determine if a feature is an actual feature or a hallucination. Hallucinations may be resolved by incorporating information about the distribution of probable images, so-called prior knowledge. The reconstructed image balances maximizing both the likelihood that explains measurements, and the prior, that is, the probability that is a valid medical image. In embodiments described herein, the diffusion models capture rich image priors from underlying data distributions. From a Bayesian perspective, the diffusion models learn the a priori probability density function of the images. Solving the Bayesian inverse problem is tantamount to drawing posterior samples (and/or computing the posterior mean) from the posterior density function that is a product of the likelihood function (physical and statistical model of the imaging system) and the learnt a priori probability density function.

Embodiments further combine the traditional plug-and-play method with the diffusion sampling framework to accurately restore complex MRI data regarding reconstruction faithfulness and perceptual quality with a limited number of neural function evaluations (NFEs). The pre-trained image denoising model is integrated as a component within an iterative optimization process to effectively remove noise (or perform other tasks such as super resolution/upscaling) from the MRI images.

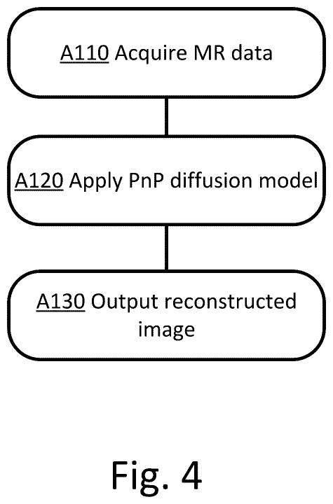

FIG. 4 depicts a method for magnetic resonance imaging and image reconstruction using denoising diffusion models for plug-and-play MR image restoration/reconstruction. The method is performed by the system of FIG. 1 or another system. The method is performed in the order shown or other orders. Additional, different, or fewer acts may be provided.

At act A110, medical imaging data of a patient is acquired. The medical imaging data may be acquired using an MRI scanning system such as described in FIG. 1. Alternatively, the medical imaging data may be provided from another source such as a database or previous scan. The medical imaging data may be acquired using an accelerated sequence. In an embodiment, an MRI system 100 acquires k-space measurements that are used to generate an initial reconstructed image that is input into the diffusion process as described below.

One limitation of MRI is acquisition time. In the example of a Knee MRI procedure, a complete acquisition may take between 15 and 20 minutes. This high acquisition time not only results in an unpleasant patient experience, as the patient is immobilized in a tube for the whole duration of the acquisition (which is not always possible for disabled people or children, for example), but also risks compromising the quality of the acquisition by increasing the risk of patient movement. It is, therefore, crucial to reduce this acquisition time as much as possible. Two acceleration methods are typically used that may be used individually or in combination. A first approach, Parallel Imaging Acceleration (PAT) involves sub-sampling the acquisition space. A second approach Simultaneous Multi-Slice (SMS) involves acquiring several slices simultaneously. An SMS factor of 2 means that two images are acquired simultaneously, and a PAT factor of 2 means that only half of the data is acquired. An acquisition that would take 20 minutes would last 5 minutes for a PAT 2 SMS 2 acquisition. Increased acceleration may result in more noise or other artifacts that need to be removed or fixed during reconstruction.

At act A120, the acquired medical imaging data is iteratively refined using diffusion PnP, wherein for each step of the iterative process, a pretrained diffusion model is used to remove noise to predict a next state of the iterative process and measurement data (G) is incorporated by solving a data proximal subproblem, the measurement data (G) applied to the next state to ensure consistency. The diffusion PnP model includes measurement during reverse diffusion steps, which is based on DDIM and supports fast sampling. This measurement is carried out after a correction step that accounts for the inaccurate estimation resulting from computing the proximal solution. As a result of this process, the medical images are restored/refined to improve the quality of the images by mitigating noise, artifacts, or missing data.

As described above, image reconstruction is an inverse problem. In an embodiment, an optimization problem for reconstruction/restoration and the prior term are separated based on an approach using the Half Quadratic Splitting (HQS) algorithm. This allows the method to solve the decoupled subproblems iteratively and leverage the diffusion sampling framework. In an embodiment, by introducing an auxiliary variable, the problem may be split into the following subproblems and be solved iteratively:

$$\begin{matrix}{z}_{k} = {arg min}_{z} \frac{1}{2 {( \sqrt{\frac{λ}{μ}} )}^{2}} {‖ z - {f}_{k} ‖}^{2} + P ( z ) \\ {f}_{k - 1} = {arg min}_{x} {‖ G - [ H ] f ‖}^{2} + μ {σ}_{n}^{2} {‖ f - {z}_{k} ‖}^{2}\end{matrix} \qquad EQUATION 1$$

Here the subproblem with the prior term is a Gaussian denoising problem, and the subproblem with the data term is a proximal operator that has a closed-form solution that depends on H. The prior term ensures that the generated sample is from the prior data distribution (here learned by a diffusion model), while the data term refines the image manifold based on the given measurement.

A fast solution may be computed based on the estimated image from the diffusion model (denoiser) for MRI IR tasks such as image denoising, image inpainting, and super-resolution. However, it may still be solved as a first-order proximal operator when there is no analytical solution, using:

$${\hat{f}}_{0}^{( t )} \approx {f}_{0}^{( t )} - \frac{{{\overline{σ}}_{t}^{2}}^{2}}{2 λ {σ}_{n}} {∇}_{{f}_{0}^{( t )}} {‖ G - [ H ] {f}_{0}^{( t )} ‖}^{2} \qquad EQUATION 2$$

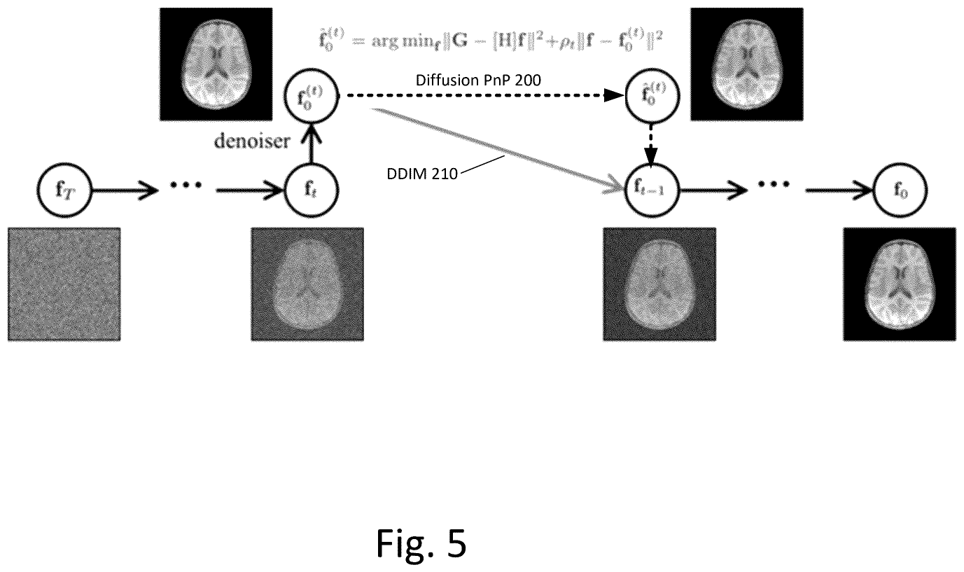

FIG. 5 depicts an example of sampling in the reverse process. After predicting the denoised image using the diffusion model for each state, the measurement is incorporated by solving the data proximal subproblem using the diffusion PnP model 200. Then, the next state is derived by adding noise back, completing one step of reverse diffusion sampling.

In an embodiment, the diffusion PnP model 200 may include aspects of or may be adapted from a Denoising Diffusion Implicit Model (DDIM). The diffusion PnP model includes measurement data for data consistency during reverse diffusion steps, which is based on DDIM and supports fast sampling. In the reverse process 210, an image is generated using the learned probability density function of contrast weighted MR image data while being constrained by a data consistency term G that represents expected/known measurements. The measurement data (G) may include measurements/linear transform of known features of the region or objects being scanned. For example, the measurement data (G) may include a ratio of the sizes or distances of or between two different features. This measurement is carried out after a correction step that accounts for the inaccurate estimation resulting from computing the proximal solution. The measurement data (G) is incorporated by solving the data proximal subproblem, for example using:

$${{f}^{′}}_{0}^{( t )} = arg {min}_{f} {‖ G - [ H ] f ‖}^{2} + {p}_{t} {‖ f - {f}_{0}^{( t )} ‖}^{2} \qquad EQUATION 3$$

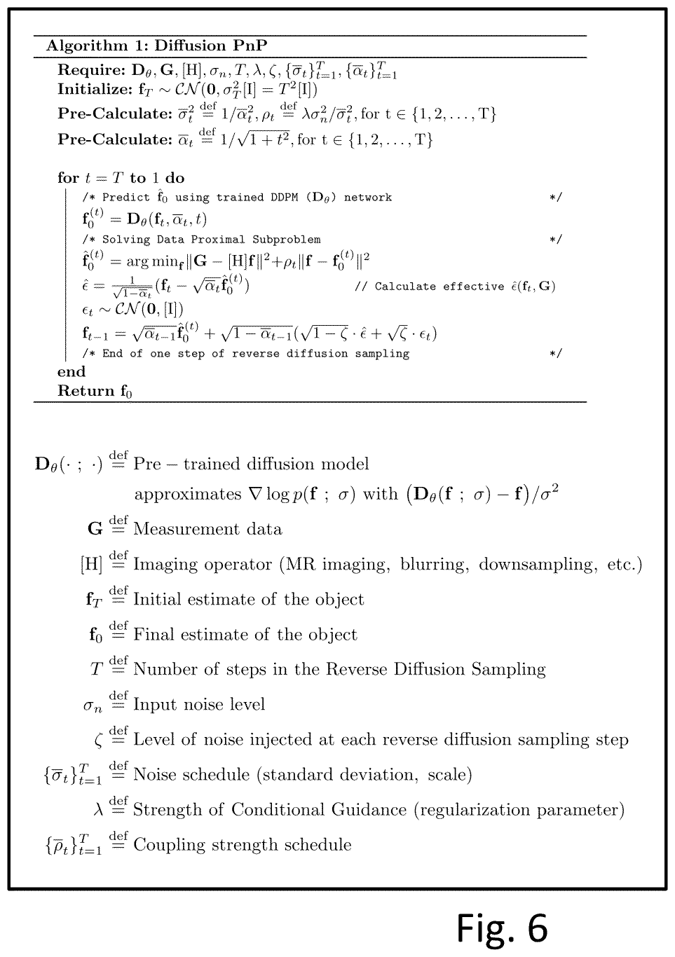

An example of an algorithm of the full method, titled diffusion PnP, is provided in FIG. 6. The algorithm combines the traditional plug-and-play method with the diffusion sampling framework to accurately restore complex MRI data regarding reconstruction faithfulness and perceptual quality. As described above, the diffusion process is split into forward and reverse diffusion processes. The forward diffusion process is a process of turning an image into noise, and the reverse diffusion process is supposed to turn that noise into the image again. The reverse process 210 starts with a noisy image. The process continuously denoises the image over and over again to steer it in a particular direction. The value T describes how many inference steps will be taken during this process. The number of steps is also referred to as Neural Function Evaluations (NFEs). The higher the value, the more steps that are taken to produce the image (also more time).

In an embodiment, the algorithm uses DDPM for the diffusion model. In an embodiment, in the reverse process, sampling is adapted from Deep Diffusion Implicit Models (DDIM). DDIM accelerates the sampling process of diffusion models by using non-Markovian diffusion processes. This approach allows for faster generation of high-quality images while maintaining the same training objective as traditional diffusion models. Implicit models focus on representing functions implicitly rather than explicitly. Instead of defining a mathematical formula directly, the implicit model defines a set of equations that describe the relationship between inputs and outputs without specifying the exact function.

The sampling process in DDIM involves sampling from the prior distribution and then iteratively sampling from the conditional distributions. This process is faster than traditional diffusion models because it does not require simulating the entire Markov chain. The number of NFEs, e.g. the total number of times the neural network needs to be called during the sampling process to generate a new image, is typically significantly lower in a DDIM compared to a standard DDPMs due to DDIM's more efficient non-Markovian diffusion process, resulting in faster generation times with fewer computations required. For example, fewer than 50 or 100 NFEs may be required to provide an acceptable output.

In an embodiment, a quadratic sampling technique is used. Sampling involves iteratively refining an image from a noisy initialization by stepping backward through a predefined sequence of time steps. The choice of these steps significantly impacts the efficiency and quality of image reconstruction. In a quadratic sampling scheme, the time steps are spaced according to a quadratic function, meaning the interval between successive steps increases quadratically as the sampling process progresses. This contrasts with uniform or geometric schedules, where the time steps are either equally spaced or decrease exponentially. The quadratic approach provides finer resolution in the early stages of denoising, when large noise components must be accurately removed, while allowing larger steps in later stages when the image structure has already stabilized. The use of this approach ensures that the early steps focus more on fine-grained denoising while later steps consolidate the reconstructed image. Different sampling techniques within diffusion models, like DPM-Solver or optimized ODE solvers, may also be used to adjust the required NFE.

In an embodiment, the PnP model is a trained neural network. In an embodiment, the network is trained to learn the inverse diffusion process, for example to progressively recover clean images from noisy versions. The training of the network may be performed at any point prior to application. The training process starts with a dataset of high-quality images that are systematically corrupted by adding noise through a series of time steps. The goal of training is to teach the model to reverse this degradation and reconstruct the original images with high fidelity. In an embodiment, the training phase follows a modified diffusion framework where the model learns to predict the noise at each step, conditioned on the noisy input. The model is trained using a loss function, for example a mean squared error (MSE) between the predicted and true noise components, ensuring that the model accurately estimates the noise distribution. By minimizing this loss across multiple training examples, the model refines its ability to denoise images across different levels of corruption.

Another key aspect of training the diffusion model for image reconstruction is the parameterization of the noise schedule. The diffusion model learns a mapping between different noise levels and the corresponding clean images, enabling it to reconstruct missing or degraded information effectively. By leveraging the learned implicit sampling trajectories, for example as adapted from DDIMs, the diffusion model may generate high-quality images with significantly fewer denoising steps compared to traditional diffusion models. Once training is complete, the diffusion model may be evaluated using test images to ensure that it generalizes well to unseen data. Metrics such as peak signal-to-noise ratio (PSNR) and structural similarity index (SSIM) may be used to assess the quality of reconstructed images. Hyperparameters such as noise schedules and network architectures may be fine-tuned to improve the performance. Through this iterative training process, the diffusion model is trained to be highly effective at reconstructing high-quality images from noisy or incomplete inputs.

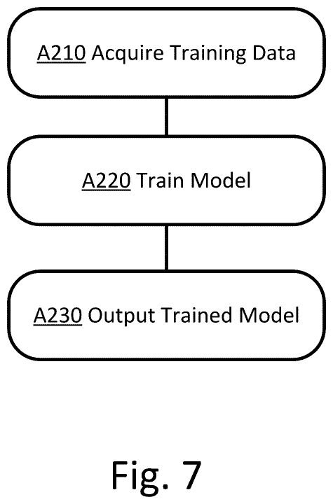

FIG. 7 depicts an example workflow for training the diffusion model. The method is performed by the system of FIG. 1 or another system. The method is performed in the order shown or other orders. Additional, different, or fewer acts may be provided.

At act A210, training data is acquired. The training data may include a dataset of medical image data that represents the style and subject matter that the diffusion model is configured to generate. Different sets of training data may be used for different models that are used for different purposes. For example, training data of the knee may be used to train a model for generating knee images, while training data of the brain may be used to train a model for generating brain images. In an example, the model was trained on a diverse dataset of around 300,000 2D images from various body regions, such as the brain, knee, and prostate. The dataset included different magnetic field strengths (1.5 T, 3 T, and 7 T) and imaging sequences like TSE, DWI, and HASTE, sourced from private and public datasets, including fastMRI. In this example, the model included a resolution of 256×256 and a total capacity of 0.7 billion parameters.

At act A220, a model to estimate a MR image is trained by finding the reverse transitions that maximize the likelihood of the training data. In an embodiment, the model is a generative model, in particular a diffusion model, for example, a DDPM or DDIM. In the learning phase, the forward process 210 learns the probability density function of MR image data by adding noise to the input image data. In the reverse process 210, an image is synthesized using the learned probability density function of MR image data. In the reverse process, a data consistency term G is used. G may include measurements/linear transform of known features of the region or objects being scanned. In an embodiment, a regularization term may be included such as subspace approaches, MP, PCA etc. on the sequence of MR images.

Different training mechanisms may be used, such as reparameterization or score-based generative modeling. In an embodiment, the diffusion model is trained in pixel space for complex-valued MRI data, employing a single diffusion prior from a diverse MR image dataset, enabling a universal framework for various inverse problems and clinical applications.

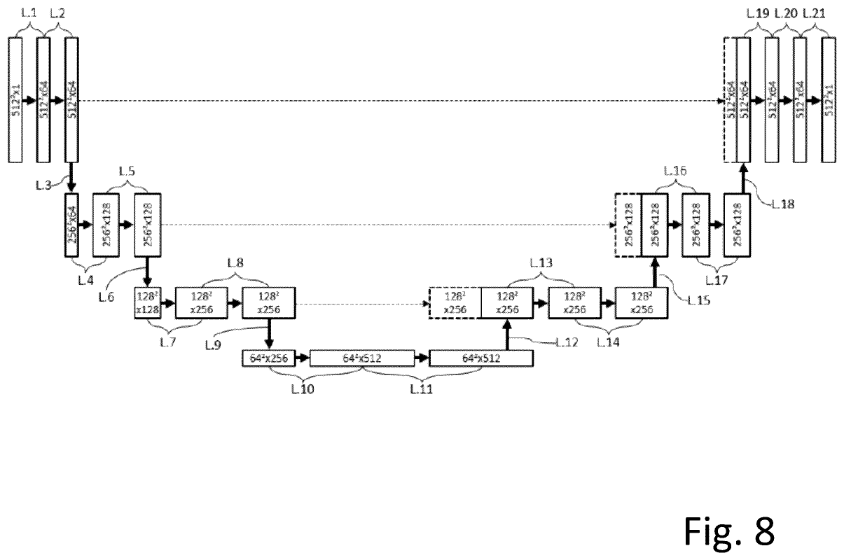

In an embodiment, the model is based on is a convolutional neural network, in particular, a convolutional neural network having a U-net structure, for example as displayed in FIG. 8. The input data to the machine learning network is a two-dimensional medical image comprising 512×512 pixel, every pixel comprising one intensity value (e.g., relating to the Hounsfield units of the respective pixels). The machine learning network comprises convolutional layers (indicated by solid, horizontal arrows), pooling layers (indicating by solid arrows pointing down), and upsampling layers (indicated by solid arrows pointing up), the number of the respective nodes is indicated within the boxes. Within the U-net structure first the input images are downsampled (decreasing the size of the images and increasing the number of channels), afterwards they are upsampled (increasing the size of the images and decreasing the number of channels) to generate a transformed image.

All except the last convolutional layers L.1, L.2, L.4, L.5, L.7, L.8, L.10, L.11, L.13, L.14, L.16, L.17, L.19, L.20 use 3×3 kernels with a padding of 1, the ReLU activation function, and a number of filters/convolutional kernels that matches the number of channels of the respective node layers as indicated in FIG. 8. The last convolutional layer uses a lxI kernel with no padding and the ReLU activation function.

The pooling layers L.3, L.6, L.9 are max-pooling layers, replacing four neighboring nodes with only one node, the value being the maximum of the values of the four neighboring nodes. The upsampling layers L.12, L.15, L.18 are transposed convolution layers with 3×3 kernels and stride 2, which effectively quadruple the number of nodes. The dashed horizontal errors correspond to concatenation operations, where the output of a convolutional layer L.2, L.5, L.8 of the downsampling branch of the U-net structure is used as additional inputs for a convolutional layer L.13, L.16, L.19 of the upsampling branch of the U-net structure. This additional input data is treated as additional channels in the input node layer for the convolutional layer L.13, L.16, L.19 of the upsampling branch.

At act A230, the trained model for denoising the MR image is output. The model may be applied to newly acquired MRI data in order to generate MR image data.

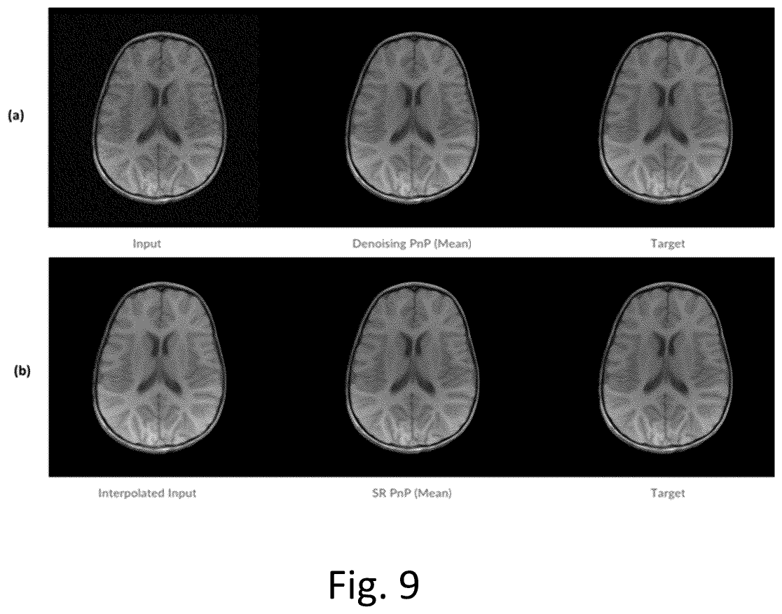

Referring back to FIG. 4, at Act A130, a reconstructed medical image of the patient is output. FIG. 9 depicts several examples of reconstructed medical images such as for (a) denoising and (b) super-resolution.

In an embodiment, the control unit 20 of the MRI system is configured for image reconstruction using denoising diffusion models for plug-and-play MR image restoration/reconstruction. The control unit 20 is in communication with a medical imaging device 36 and a server (not shown). The control unit includes a processor, a memory, and an interface. The medical imaging device is configured to acquire MR imaging data, for example k-space data that is reconstructed into an MR image of an organ or region of a patient. The reconstructed image may be a two-dimensional distribution of pixels representing an area of the patient and/or a three-dimensional distribution of voxels representing a volume of the patient. The processor is configured to implement diffusion models configured to output MR images when input the MR imaging data. The diffusion models may input a noisy image and denoise the image. The diffusion model may input an image and upscale the image and/or attempt to recover a degraded image. The memory 24 is configured to store instructions and the parameters for the model(s). The interface 26 is configured to display the reconstructed/restored images and/or accept inputs from a user. The server may perform similar tasks as the control unit and/or may provide some additional processing, storage, or analysis for example using a cloud based platform.

In an embodiment, the medical imaging device is an MR imaging device 36, for example, as described above in FIG. 1. The MR system 100 of FIG. 1 includes an MR scanner 36 or system, a computer based on data obtained by MR scanning, a server, or another processor 22. The MR imaging device 36 is only exemplary, and a variety of MR scanning systems may be used to collect the MR data. The MR imaging device 36 (also referred to as a MR scanner or image scanner) is configured to scan a patient 11. The MR imaging device 36 scans a patient 11 to provide k-space measurements (measurements in the frequency domain).

The processor may include an image processor that generates images using a machine learning network (machine learning model). The image processor is a general processor, digital signal processor, three-dimensional data processor, graphics processing unit, application specific integrated circuit, field programmable gate array, artificial intelligence processor, digital circuit, analog circuit, combinations thereof, or another now known or later developed device for image generation. The image processor is a single device, a plurality of devices, or a network. For more than one device, parallel or sequential division of processing may be used. Different devices making up the image processor may perform different functions. In one embodiment, the image processor is also a control processor or other processor of the imaging device. Other image processors of the imaging device or external to the imaging device may be used. The image processor is configured by software, firmware, and/or hardware to process the data acquired by the imaging device and output one or more images.

The instructions for implementing the processes, methods, and/or techniques discussed herein are provided on non-transitory computer-readable storage media or memories, such as a cache, buffer, RAM, removable media, hard drive, or other computer readable storage media, for example the memory. The instructions are executable by the processor or another processor. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the instructions set, storage media, processor or processing strategy and may be performed by software, hardware, integrated circuits, firmware, micro code, and the like, operating alone or in combination. In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. Because some of the constituent system components and method steps depicted in the accompanying figures may be implemented in software, the actual connections between the system components (or the process steps) may differ depending upon the manner in which the present embodiments are programmed.

In an embodiment, the processor 22 implements one or more machine learning networks that are stored in the memory. In general, a trained machine learning network mimics cognitive functions that humans associate with other human minds. In particular, by training based on training data the machine learning network is able to adapt to new circumstances and to detect and extrapolate patterns. Another term for “trained machine learning network” is “trained function”. In general, parameters of a machine learning network can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning networks can be adapted iteratively by several steps of training. In particular, within the training a certain cost function can be minimized. In particular, within the training of a neural network the backpropagation algorithm can be used. In particular, a machine learning network may comprise a neural network, a support vector machine, a decision tree and/or a Bayesian network, and/or the machine learning network can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network, and/or a generative adversarial network.

In an embodiment, the processor 22 implements a diffusion process for training and configuring the model. The diffusion process includes forward diffusion and reverse diffusion. Forward diffusion is used to add noise to the input image using a schedule which determines how much noise is added at the given step t. Reverse diffusion consists of multiple steps in which a small amount of noise is removed at every step. In an embodiment, the diffusion models use a modified U-Net architecture, for example as described above in FIG. 8. In an embodiment, the model(s) are provided by or implemented with a neural network trained using deep learning. The network(s) may be defined as a plurality of sequential feature units or layers. Sequential is used to indicate the general flow of output feature values from one layer to input to a next layer. The information from the next layer is fed to a next layer, and so on until the final output. The layers may only feed forward or may be bi-directional, including some feedback to a previous layer. The nodes of each layer or unit may connect with all or only a sub-set of nodes of a previous and/or subsequent layer or unit. Skip connections may be used, such as a layer outputting to the sequentially next layer as well as other layers. Rather than pre-programming the features and trying to relate the features to attributes, the deep architecture is defined to learn the features at different levels of abstraction the input data. The features are learned to reconstruct lower level features (i.e., features at a more abstract or compressed level). For example, features for generating a fused image or higher resolution image are learned. For a next unit, features for reconstructing the features of the previous unit are learned, providing more abstraction. Each node of the unit represents a feature. Different units are provided for learning different features.

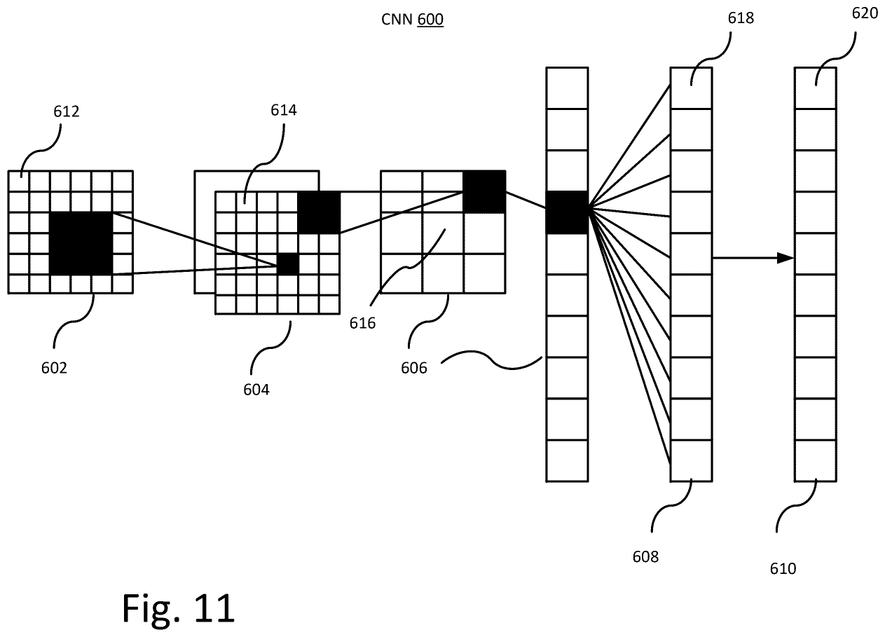

Various units or layers may be used, such as convolutional, pooling (e.g., max-pooling), deconvolutional, fully connected, or other types of layers. Within a unit or layer, any number of nodes is provided. For example, 100 nodes are provided. Later or subsequent units may have more, fewer, or the same number of nodes. In general, for convolution, subsequent units have more abstraction. FIG. 11 shows an embodiment of an artificial neural network (ANN) 500, in accordance with one or more embodiments. Alternative terms for “artificial neural network” are “neural network”, “artificial neural net” or “neural net”. The artificial neural network 500 may be used in part in, for example, the one or more machine learning based networks utilized for the first generative model 400 and/or second generative model 700, etc.

The artificial neural network 500 includes nodes 502-522 and edges 532, 534, . . . , 536, wherein each edge 532, 534, . . . , 536 is a directed connection from a first node 502-522 to a second node 502-522. In general, the first node 502-522 and the second node 502-522 are different nodes 502-522, it is also possible that the first node 502-522 and the second node 502-522 are identical. For example, in FIG. 11, the edge 532 is a directed connection from the node 502 to the node 506, and the edge 534 is a directed connection from the node 504 to the node 506. An edge 532, 534, . . . , 536 from a first node 502-522 to a second node 502-522 is also denoted as “ingoing edge” for the second node 502-522 and as “outgoing edge” for the first node 502-522.

In this embodiment, the nodes 502-522 of the artificial neural network 500 may be arranged in layers 524-530, wherein the layers may include an intrinsic order introduced by the edges 532, 534, . . . , 536 between the nodes 502-522. In particular, edges 532, 534, . . . , 536 may exist only between neighboring layers of nodes. In the embodiment shown in FIG. 11, there is an input layer 524 including only nodes 502 and 504 without an incoming edge, an output layer 530 including only node 522 without outgoing edges, and hidden layers 526, 528 in-between the input layer 524 and the output layer 530. In general, the number of hidden layers 526, 528 may be chosen arbitrarily. The number of nodes 502 and 504 within the input layer 524 usually relates to the number of input values of the neural network 500, and the number of nodes 522 within the output layer 530 usually relates to the number of output values of the neural network 500.

In particular, a (real) number may be assigned as a value to every node 502-522 of the neural network 500. Here, x⁽ⁿ⁾ᵢ denotes the value of the i-th node 502-522 of the n-th layer 524-530. The values of the nodes 502-522 of the input layer 524 are equivalent to the input values of the neural network 500, the value of the node 522 of the output layer 530 is equivalent to the output value of the neural network 500. Furthermore, each edge 532, 534, . . . , 536 may include a weight being a real number, in particular, the weight is a real number within the interval [−1, 1] or within the interval [0, 1]. Here, w^{(m,n)}_{i,j} denotes the weight of the edge between the i-th node 502-522 of the m-th layer 524-530 and the j-th node 502-522 of the n-th layer 524-530. Furthermore, the abbreviation w⁽ⁿ⁾_{i,j} is defined for the weight w^{(n,m+1)}_{i,j}.

In particular, to calculate the output values of the neural network 500, the input values are propagated through the neural network. In particular, the values of the nodes 502-522 of the (n+1)-th layer 524-530 may be calculated based on the values of the nodes 502-522 of the n-th layer 524-530 by

$${x}_{j}^{( n + 1 )} = f ( {\sum }_{i} {x}_{i}^{( n )} \cdot {w}_{i , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 524 are given by the input of the neural network 500, wherein values of the first hidden layer 526 may be calculated based on the values of the input layer 524 of the neural network, wherein values of the second hidden layer 528 may be calculated based in the values of the first hidden layer 526, etc.

In order to set the values w^{(m,n)}_{i,j} for the edges, the neural network 500 has to be trained using training data. In particular, training data includes training input data and training output data (denoted as tᵢ). For a training step, the neural network 500 is applied to the training input data to generate calculated output data. In particular, the training data and the calculated output data include a number of values, said number being equal with the number of nodes of the output layer.

In particular, a comparison between the calculated output data and the training data is used to recursively adapt the weights within the neural network 500 (backpropagation algorithm). In particular, the weights are changed according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate, and the numbers δ⁽ⁿ⁾ⱼ may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on δ⁽ⁿ⁺¹⁾ⱼ, if the (n+1)-th layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 530, wherein f′ is the first derivative of the activation function, and y⁽ⁿ⁺¹⁾ⱼ, is the comparison training value for the j-th node of the output layer 530.

FIG. 12 shows a convolutional neural network (CNN) 600, in accordance with one or more embodiments. Machine learning networks described herein, such as, e.g., the first generative model 400 and/or second generative model 700 etc. may be implemented using convolutional neural network 600.

In the embodiment shown in FIG. 12 the convolutional neural network includes 600 an input layer 602, a convolutional layer 604, a pooling layer 606, a fully connected layer 608, and an output layer 610. Alternatively, the convolutional neural network 600 may include several convolutional layers 604, several pooling layers 606, and several fully connected layers 608, as well as other types of layers. The order of the layers may be chosen arbitrarily, usually fully connected layers 608 are used as the last layers before the output layer 610.

In particular, within a convolutional neural network 600, the nodes 612-620 of one layer 602-610 may be considered to be arranged as a d-dimensional matrix or as a d-dimensional image. In particular, in the two-dimensional case the value of the node 612-620 indexed with i and j in the n-th layer 602-610 may be denoted as x⁽ⁿ⁾_{[i,j]}. However, the arrangement of the nodes 612-620 of one layer 602-610 does not have an effect on the calculations executed within the convolutional neural network 600 as such, since these are given solely by the structure and the weights of the edges.

In particular, a convolutional layer 604 is characterized by the structure and the weights of the incoming edges forming a convolution operation based on a certain number of kernels. In particular, the structure and the weights of the incoming edges are chosen such that the values x⁽ⁿ⁾ₖ of the nodes 614 of the convolutional layer 604 are calculated as a convolution x⁽ⁿ⁾ₖ=Kₖ*x⁽ⁿ⁻¹⁾ based on the values x⁽ⁿ⁻¹⁾ of the nodes 612 of the preceding layer 602, where the convolution * is defined in the two-dimensional case as:

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

Here the k-th kernel Kₖ is a d-dimensional matrix (in this embodiment a two-dimensional matrix), which is usually small compared to the number of nodes 612-618 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 612-620 in the respective layer 602-610. In particular, for a convolutional layer 604, the number of nodes 614 in the convolutional layer is equivalent to the number of nodes 612 in the preceding layer 602 multiplied with the number of kernels.

If the nodes 612 of the preceding layer 602 are arranged as a d-dimensional matrix, using a plurality of kernels may be interpreted as adding a further dimension (denoted as “depth” dimension), so that the nodes 614 of the convolutional layer 604 are arranged as a (d+1)-dimensional matrix. If the nodes 612 of the preceding layer 602 are already arranged as a (d+1)-dimensional matrix including a depth dimension, using a plurality of kernels may be interpreted as expanding along the depth dimension, so that the nodes 614 of the convolutional layer 604 are arranged also as a (d+1)-dimensional matrix, wherein the size of the (d+1)-dimensional matrix with respect to the depth dimension is by a factor of the number of kernels larger than in the preceding layer 602.

The advantage of using convolutional layers 604 is that spatially local correlation of the input data may exploited by enforcing a local connectivity pattern between nodes of adjacent layers, in particular by each node being connected to only a small region of the nodes of the preceding layer.

In the embodiment shown in FIG. 12, the input layer 602 includes 36 nodes 612, arranged as a two-dimensional 6×6 matrix. The convolutional layer 604 includes 72 nodes 614, arranged as two two-dimensional 6×6 matrices, each of the two matrices being the result of a convolution of the values of the input layer with a kernel. Equivalently, the nodes 614 of the convolutional layer 604 may be interpreted as arranges as a three-dimensional 6×6×2 matrix, wherein the last dimension is the depth dimension.

A pooling layer 606 may be characterized by the structure and the weights of the incoming edges and the activation function of its nodes 616 forming a pooling operation based on a non-linear pooling function f. For example, in the two dimensional case the values x⁽ⁿ⁾ of the nodes 616 of the pooling layer 606 may be calculated based on the values x⁽ⁿ⁻¹⁾ of the nodes 614 of the preceding layer 604 as

$${x}^{( n )} [ i , 1 ] = f ( {x}^{( n - 1 )} [ {id}_{1} , {jd}_{2} ] , … , {x}^{( n - 1 )} [ i {d}_{1} + {d}_{1} - 1 , j {d}_{2} + {d}_{2} - 1 ] )$$

In other words, by using a pooling layer 606, the number of nodes 614, 616 may be reduced, by replacing a number d1·d2 of neighboring nodes 614 in the preceding layer 604 with a single node 616 being calculated as a function of the values of said number of neighboring nodes in the pooling layer. In particular, the pooling function f may be the max-function, the average, or the L2-Norm. In particular, for a pooling layer 606 the weights of the incoming edges are fixed and are not modified by training.

The advantage of using a pooling layer 606 is that the number of nodes 614, 616 and the number of parameters is reduced. This leads to the amount of computation in the network being reduced and to a control of overfitting.

In the embodiment shown in FIG. 12, the pooling layer 606 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 608 may be characterized by the fact that a majority, in particular, all edges between nodes 616 of the previous layer 606 and the nodes 618 of the fully-connected layer 608 are present, and wherein the weight of each of the edges may be adjusted individually.

In this embodiment, the nodes 616 of the preceding layer 606 of the fully-connected layer 608 are displayed both as two-dimensional matrices, and additionally as non-related nodes (indicated as a line of nodes, wherein the number of nodes was reduced for a better presentability). In this embodiment, the number of nodes 618 in the fully connected layer 608 is equal to the number of nodes 616 in the preceding layer 606. Alternatively, the number of nodes 616, 618 may differ.

A convolutional neural network 600 may also include a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks may be wired using summation (residual/dense neural networks), element-wise multiplication (attention) or other differentiable operators. Therefore, the convolutional neural network architecture may be nested rather than being sequential if the whole pipeline is differentiable.

In particular, convolutional neural networks 600 may be trained based on the backpropagation algorithm. For preventing overfitting, methods of regularization may be used, e.g. dropout of nodes 612-620, stochastic pooling, use of artificial data, weight decay based on the L1 or the L2 norm, or max norm constraints. Different loss functions may be combined for training the same neural network to reflect the joint training objectives. A subset of the neural network parameters may be excluded from optimization to retain the weights pretrained on another datasets.

The operator interface 26 includes an input device and an output device. The input may be an interface, such as interfacing with a computer network, memory, database, medical image storage, or other source of input data. The input may be a user input device, such as a mouse, trackpad, keyboard, roller ball, touch pad, touch screen, or another apparatus for receiving user input. The output is a display device but may be an interface. The reconstructed/restored images are displayed. For example, a high resolution image of a region of the patient 11 is displayed. The display is a CRT, LCD, plasma, projector, printer, or other display device. The display is configured by loading an image to a display plane or buffer. The display is configured to display the image of the region of the patient 11. The operator interface may include a graphical user interface (GUI) enabling user interaction with the medical imaging device and enables user modification or selections in substantially real time.

While the invention has been described above by reference to various embodiments, many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

The following is a list of non-limiting illustrative embodiments disclosed herein:

Illustrative embodiment 1: A method for diffusion plug and play (PnP) image reconstruction of medical imaging data, the method comprising: acquiring medical imaging data of a portion of a patient; iteratively refining the medical imaging data using diffusion PnP, wherein for each step of an iterative process, a pretrained diffusion model is used to remove noise to predict a next state of the iterative process and measurement data is incorporated by solving a data proximal subproblem, the measurement data applied to the next state to ensure consistency; and outputting a reconstructed medical image of the portion of the patient.

Illustrative embodiment 2. The method of Illustrative embodiment 1, wherein the data proximal subproblem is solved by the following equation:

$${{f}^{′}}_{0}^{( t )} = arg {min}_{f} {‖ G - [ H ] f ‖}^{2} + {p}_{t} {‖ f - {f}_{0}^{( t )} ‖}^{2}$$

where G=the measurement data.

Illustrative embodiment 3. The method of Illustrative embodiment 1, wherein the measurement data comprises measurements and/or linear transform of known features of the portion being imaged.

Illustrative embodiment 4. The method of Illustrative embodiment 1, wherein the pretrained diffusion model adapts a quadratic sequence from a Denoising Diffusion Implicit Model (DDIM) for determining sampling.

Illustrative embodiment 5. The method of Illustrative embodiment 4, wherein there are more sampling steps at low-noise regions than high-noise regions.

Illustrative embodiment 6. The method of Illustrative embodiment 1, wherein the medical imaging data is acquired using magnetic resonance imaging.

Illustrative embodiment 7. The method of Illustrative embodiment 6, wherein the medical imaging data is undersampled k-space data.

Illustrative embodiment 8. The method of Illustrative embodiment 1, wherein the pretrained diffusion model comprises a trained Denoising Diffusion Probabilistic Model (DDPM).

Illustrative embodiment 9. The method of Illustrative embodiment 1, wherein fewer than 100 neural function evaluations are used by the pretrained diffusion model.

Illustrative embodiment 10. The method of Illustrative embodiment 8, further comprising: tuning diffusion PnP hyperparameters that control a strength of the condition guidance and/or a level of noise injected at each timestep of the iterative refinement by the y the pretrained diffusion model.

Illustrative embodiment 11. A system for diffusion plug and play (PnP) image reconstruction of magnetic resonance (MR) data, the system comprising: a medical imaging device configured to acquire MR data; a memory configured to store a model configured to reconstruct an MR images when input MR data, wherein the model is configured to iteratively refine the MR data using diffusion PnP, wherein for each step of the iterative process, a pretrained diffusion model is used to remove noise to predict a next state of the iterative process and measurement data is incorporated by solving a data proximal subproblem, the measurement data applied to the next state to ensure consistency; and a processor configured to reconstruct and/or restore the MR image from the MR data using the model.

Illustrative embodiment 12. The system of Illustrative embodiment 11, wherein the data proximal subproblem is solved by the following equation:

$${{f}^{′}}_{0}^{( t )} = arg {min}_{f} {‖ G - [ H ] f ‖}^{2} + {p}_{t} {‖ f - {f}_{0}^{( t )} ‖}^{2}$$

where G=the measurement data.

Illustrative embodiment 13. The system of Illustrative embodiment 11, wherein the measurement data comprises measurements and/or linear transform of known features of a portion being imaged.

Illustrative embodiment 14. The system of Illustrative embodiment 11, wherein the pretrained diffusion model adapts a quadratic sequence from a Denoising Diffusion Implicit Model (DDIM) for determining sampling.

Illustrative embodiment 15. The system of Illustrative embodiment 14, wherein there are more sampling steps at low-noise regions.

Illustrative embodiment 16. The system of Illustrative embodiment 11, wherein the medical imaging data is undersampled k-space data.

Illustrative embodiment 17. The system of Illustrative embodiment 11, wherein the pretrained diffusion model comprises a trained Denoising Diffusion Probabilistic Model (DDPM).

Illustrative embodiment 18. The system of Illustrative embodiment 11, wherein fewer than 100 neural function evaluations are used by the pretrained diffusion model.

Illustrative embodiment 19. A method for diffusion plug and play (PnP) image restoration of medical imaging data, comprising: acquiring medical imaging data; iteratively restoring the medical imaging data using a diffusion PnP model that includes measurement during reverse diffusion steps, wherein the measurement is carried out after a correction step that accounts for an inaccurate estimation resulting from computing a proximal solution; and outputting a restored image.

Illustrative embodiment 20. The method of Illustrative embodiment 19, wherein the diffusion PnP model is based on a Denoising Diffusion Implicit Model and supports fast sampling.

1. A method for diffusion plug and play (PnP) image reconstruction of medical imaging data, the method comprising:
acquiring medical imaging data of a portion of a patient;
iteratively refining the medical imaging data using diffusion PnP, wherein for each step of an iterative process, a pretrained diffusion model is used to remove noise to predict a next state of the iterative process and measurement data is incorporated by solving a data proximal subproblem, the measurement data applied to the next state to ensure consistency; and
outputting a reconstructed medical image of the portion of the patient.

2. The method of claim 1, wherein the data proximal subproblem is solved by the following equation:
$${{f}^{′}}_{0}^{( t )} = arg {min}_{f} {‖ G - [ H ] f ‖}^{2} + {p}_{t} {‖ f - {f}_{0}^{( t )} ‖}^{2}$$
where G=the measurement data.

3. The method of claim 1, wherein the measurement data comprises measurements and/or linear transform of known features of the portion being imaged.

4. The method of claim 1, wherein the pretrained diffusion model adapts a quadratic sequence from a Denoising Diffusion Implicit Model (DDIM) for determining sampling.

5. The method of claim 4, wherein there are more sampling steps at low-noise regions than high-noise regions.

6. The method of claim 1, wherein the medical imaging data is acquired using magnetic resonance imaging.

7. The method of claim 6, wherein the medical imaging data is undersampled k-space data.

8. The method of claim 1, wherein the pretrained diffusion model comprises a trained Denoising Diffusion Probabilistic Model (DDPM).

9. The method of claim 1, wherein fewer than 100 neural function evaluations are used by the pretrained diffusion model.

10. The method of claim 8, further comprising:
tuning diffusion PnP hyperparameters that control a strength of the condition guidance and/or a level of noise injected at each timestep of the iterative refinement by the y the pretrained diffusion model.

11. A system for diffusion plug and play (PnP) image reconstruction of magnetic resonance (MR) data, the system comprising:
a medical imaging device configured to acquire MR data;
a memory configured to store a model configured to reconstruct an MR images when input MR data, wherein the model is configured to iteratively refine the MR data using diffusion PnP, wherein for each step of the iterative process, a pretrained diffusion model is used to remove noise to predict a next state of the iterative process and measurement data is incorporated by solving a data proximal subproblem, the measurement data applied to the next state to ensure consistency; and
a processor configured to reconstruct and/or restore the MR image from the MR data using the model.

12. The system of claim 11, wherein the data proximal subproblem is solved by the following equation:
$${{f}^{′}}_{0}^{( t )} = arg {min}_{f} {‖ G - [ H ] f ‖}^{2} + {p}_{t} {‖ f - {f}_{0}^{( t )} ‖}^{2}$$
where G=the measurement data.

13. The system of claim 11, wherein the measurement data comprises measurements and/or linear transform of known features of a portion being imaged.

14. The system of claim 11, wherein the pretrained diffusion model adapts a quadratic sequence from a Denoising Diffusion Implicit Model (DDIM) for determining sampling.

15. The system of claim 14, wherein there are more sampling steps at low-noise regions.

16. The system of claim 11, wherein the medical imaging data is undersampled k-space data.

17. The system of claim 11, wherein the pretrained diffusion model comprises a trained Denoising Diffusion Probabilistic Model (DDPM).

18. The system of claim 11, wherein fewer than 100 neural function evaluations are used by the pretrained diffusion model.

19. A method for diffusion plug and play (PnP) image restoration of medical imaging data, comprising:
acquiring medical imaging data;
iteratively restoring the medical imaging data using a diffusion PnP model that includes measurement during reverse diffusion steps, wherein the measurement is carried out after a correction step that accounts for an inaccurate estimation resulting from computing a proximal solution; and
outputting a restored image.

20. The method of claim 19, wherein the diffusion PnP model is based on a Denoising Diffusion Implicit Model and supports fast sampling.
